(19) United States
(12) Patent Application Publication
(10) Pub. No.: US 2015/0201628 A1
(43) Pub. Date: Jul. 23, 2015

(54) SYSTEM AND METHOD TO RESTRAIN AND PROVIDE AN INHALANT AGENT TO AN ANIMAL

(71) Applicant: PULSE NEEDLEFREE SYSTEMS, INC., LENEXA, KS (US)

(72) Inventors: Mikhail A. ZOLOTUKHIN, Shawnee, KS (US); Curtis A. CIVIS, Liberal, KS (US)

(73) Assignee: PULSE NEEDLEFREE SYSTEMS, INC., LENEXA, KS (US)

(21) Appl. No.: 14/601,770

(22) Filed: Jan. 21, 2015

Related U.S. Application Data
(60) Provisional application No. 61/929,641, filed on Jan. 21, 2014.

(51) Int. Cl. A22B 3/00; A61M 16/20; A61M 16/10; A61M 16/12; A22B 1/00; A61M 16/06

(52) U.S. Cl. CPC A22B 3/005; A22B 1/00; A61M 16/0683; A61M 16/104; A61M 16/122; A61M 16/20; A61M 2202/0241; A61M 2250/00

(57) ABSTRACT

An inhaled agent administration system for animals that restrains the animal and provides a gas to the animal through a hood. The inhaled agent administration system includes an animal restraint system, a hood enclosure to cover the animal's head, and a gas supply system. A method for using the inhaled agent administration system is also disclosed.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND

In livestock production operations, there is often a need to euthanize sick or injured animals because they are in pain, are no longer productive, carry diseases which place other animals at risk, or will not be appropriate for slaughter at a meat processing facility. Common methods of euthanasia include manually applied blunt force trauma to the head, gunshot, captive bolt guns, exsanguination, immersion into a chamber containing gases to be inhaled by the animal, and, less frequently, parenteral injection of a euthanizing agent. Each of these methods has significant drawbacks. Personnel must be trained in the proper use of such methods and many of these methods present risk of injury to the farm worker in the event of human error or other accident. Also, if not implemented correctly, these methods can be ineffective and leave the animal in a prolonged state of suffering.

Furthermore, in livestock production systems many painful or difficult animal husbandry tasks are currently performed without anesthesia. To be effective, an anesthesia system would need to be simple and functional for personnel without advanced veterinary training.

Many anesthesia or euthanasia methods cannot be implemented in commercial farm operations because the skill of the workers is not sufficient for the particular method. For example, while parenteral injection of an anesthetizing or euthanizing agent may be effective, it is not practical for the farm personnel to administer such products on a routine basis and the risk of intentional or unintentional misuse are severe. There is also increasing public and consumer scrutiny of on-farm animal husbandry practices. Some euthanasia methods, such as blunt force trauma with captive bolt equipment and exsanguination, are viewed as inhumane by the public and consumers.

### BRIEF SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Utilizing the methods and systems described herein, an inhaled agent administration system is provided having a restraint system, a hood enclosure that covers the head of the animal without requiring dis-engagement of the restraint system, and a gas supply system to provide a gas into the hood enclosure.

One aspect of the invention is directed to an inhaled agent administration system for providing a gas to an animal, comprising an animal restraint system to minimize animal movement; a hood enclosure configured to cover the animal's head having a first open position and a second closed position and wherein the hood enclosure is configured to allow unencumbered use of the animal restraint system when in the first open position; and a gas supply system in fluid communication with the hood enclosure; and wherein the hood enclosure is configured to restrict release of a gas when the hood enclosure is in the second closed position.

In one aspect of the invention, the hood enclosure is configured to be moved from the first open position to the second closed position without interfering with the animal restraint system.

In one aspect of the invention, the animal restraint system comprises a snare configured to interface with the animal's mouth, snout or both the mouth and snout. In another aspect of the invention, the animal restraint system comprises a snare configured to interface with the animal's head or neck.

In one aspect of the invention, the gas supply system comprises a gas supply tube opening into the hood enclosure. In one aspect of the invention, the gas supply system may comprise a portable gas tank reservoir and the gas supply tube connects the reservoir to the hood enclosure. In one aspect of the invention, he gas supply system comprises a timer to control the supply gas for a specified period of time.

Another aspect of the invention is directed to a method of anesthetizing or euthanizing an animal, the method comprising the steps of physically restraining the animal with an animal restraint system; moving a hood enclosure from a first open position to a second closed position over the animal's head; supplying a gas into the hood enclosure through a gas supply system in a sufficient amount to anesthetize or euthanize the animal.

In one such aspect of the invention, the supplying step may comprise supplying the gas for a specified period of time.

In one aspect of the invention, the hood enclosure is attached to the animal restraint system.

Another aspect of the invention is directed to a method of providing medication to an animal, the method comprising the steps of physically restraining the animal with an animal restraint system; moving a hood enclosure from a first open position to a second closed position over the animal's head; and supplying a gas into the hood enclosure through a gas supply system in a specified amount.

### BRIEF DESCRIPTION OF THE SEVERAL VIEWS OF THE DRAWINGS

The present invention is described in detail below with reference to the attached drawing figures.

FIG. 1 is a perspective view of an inhaled agent administration system in accordance with an embodiment of the present invention, with the hood enclosure in the first open position.

FIG. 2 is a perspective view of an inhaled agent administration system in accordance with an embodiment of the present invention, with the hood enclosure in the second closed position.

FIG. 3 is a perspective view of an inhaled agent administration system in accordance with an embodiment of the present invention, with the animal restraint system engaged and the hood enclosure in the first open position.

FIG. 4 is a perspective view of an inhaled agent administration system in accordance with an embodiment of the present invention, with the animal restraint system engaged and the hood enclosure in the second closed position.

FIG. 5 is a perspective view of an inhaled agent administration system in accordance with an embodiment of the present invention, with the animal restraint system engaged and the hood enclosure in the second closed position.

FIG. 6 is a side view of a gas supply system in accordance with one embodiment of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies.

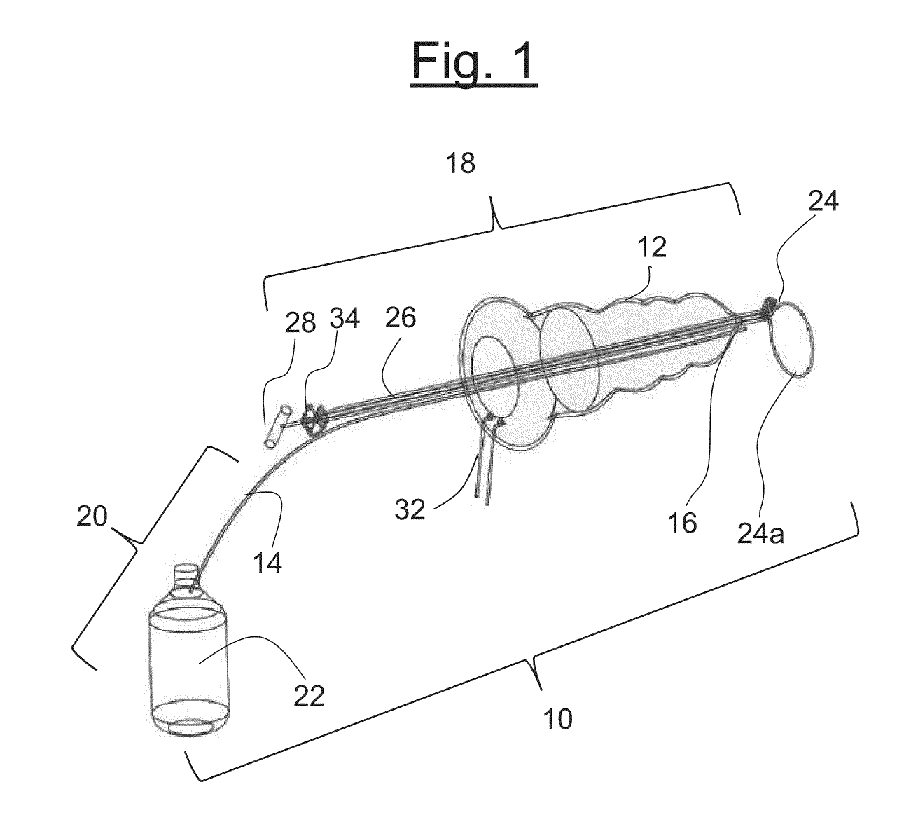

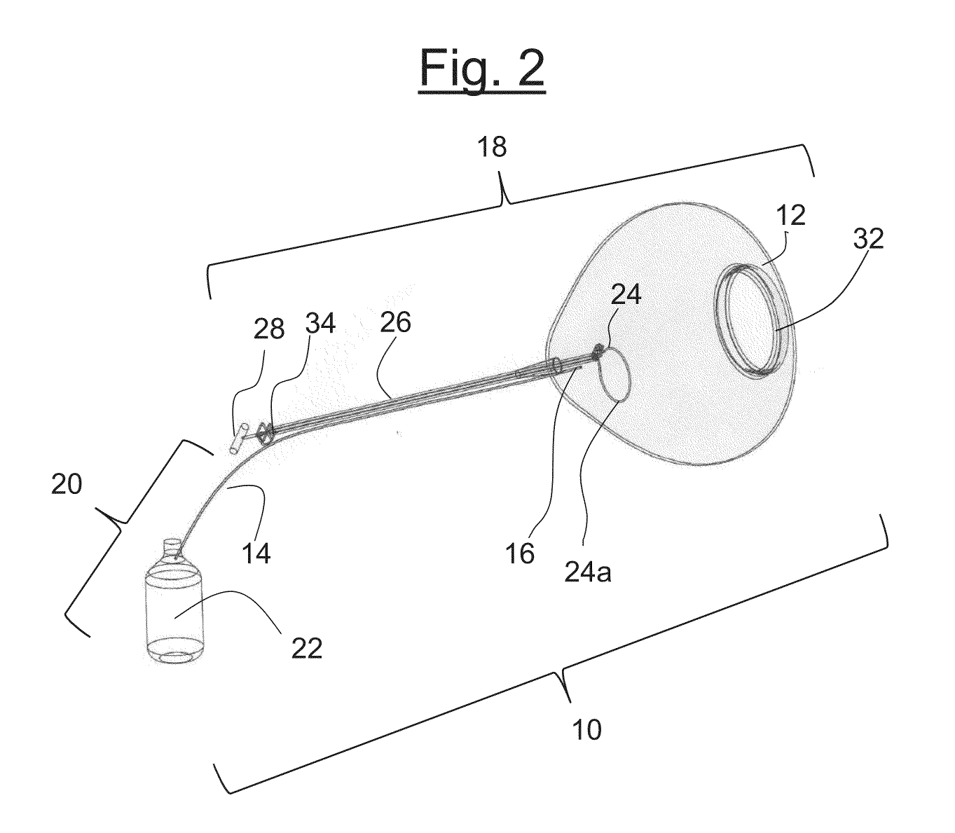

FIG. 1 depicts an exemplary embodiment of an inhaled agent administration system 10 for anesthetizing or euthanizing an animal 30 consistent with the present invention. The inhaled agent administration system 10 includes a hood enclosure 12 to receive a gas by means of a gas supply tube 14 through an opening 16 in the hood enclosure 12. The hood enclosure 12 is attached to an animal restraint system 18 to allow the operator to immobilize or restrain the animal 30. The hood enclosure 12 is movable from a first open position, as depicted in FIG. 1, to a second closed position, in which hood enclosure 12 can be positioned over the head of the animal 30, as depicted in FIG. 2. Returning to FIG. 1, in the first open position, the hood enclosure surrounds the pole 26 of the animal restraint system 18, to allow unencumbered operation of the animal restraint system 18 in restraining the animal 30. Although the embodiment depicted in FIG. shows a hood enclosure 12 attached to animal restraint system 18, it should be understood that hood enclosure 12 and animal restraint system need not be physically attached, but may be configured in any relationship that allows the user unencumbered operation of the animal restraint system when the hood enclosure 12 is in a first open position.

In the second closed position depicted in FIG. 2, the hood enclosure 12 is positioned distally towards the animal 30 and is secured around the head or neck of the animal 30, while the animal restraint system 18 is engaged to control movement of the animal 30. In the second closed position, the hood enclosure 12 creates a pocket of air or gas between the animal 30 and the hood enclosure 12. The hood enclosure 12 may be secured around the head or neck of the animal 30 by a cord 32. Alternatively other types of fasteners or retainers may be used, such as a belt or any other means of connection that are common and known in the art. Cord 32 restricts the release of gas from the hood enclosure 12 when the hood enclosure 12 is in the closed, or secured, position by being firmly fit around the head or neck of the animal 30. The positioning of the hood enclosure 12 allows the operator to maintain his or her grip on the animal restraint system 18 and use the animal restraint system 18 to control movement of the animal 30 while placing the hood enclosure 12 over the head of the animal 30.

FIG. 1 also depicts the gas supply system 20 for the inhaled agent administration system. The gas supply system 20 includes a portable gas reservoir 22, a gas supply tube 14 and an opening 16 in the hood enclosure 12. The gas reservoir 22 may be a canister, such as a portable liquid carbon dioxide storage canister, a plumbed supply system or any other means of holding a quantity of gas. When gas is transferred from the gas reservoir 22 to the gas supply tube 14, such as by opening a valve or otherwise allowing gas to pass from gas reservoir 22 into tube 14, the gas enters the hood enclosure 12 through the opening 16. The gas supply tube 14 may be unattached to the animal restraint system 18, attached to the exterior of the animal restraint system 18 or held within the animal restraint system 18 by a hollow opening in the pole 26 of the animal restraint system 18. Gas is held within the hood enclosure 12 by a tensioned fastener, which may include a firm-fitting cord 32 surrounding the hood enclosure 12. In the embodiment depicted in the Figures, cord 32 surrounds the opening of hood enclosure 12. The hood enclosure 12 may be rigid or flexible. In one embodiment, the hood enclosure 12 is made of a polyvinyl chloride material to retain the gas within the hood enclosure 12 without significant seepage of the gas from the material comprising the hood enclosure 12.

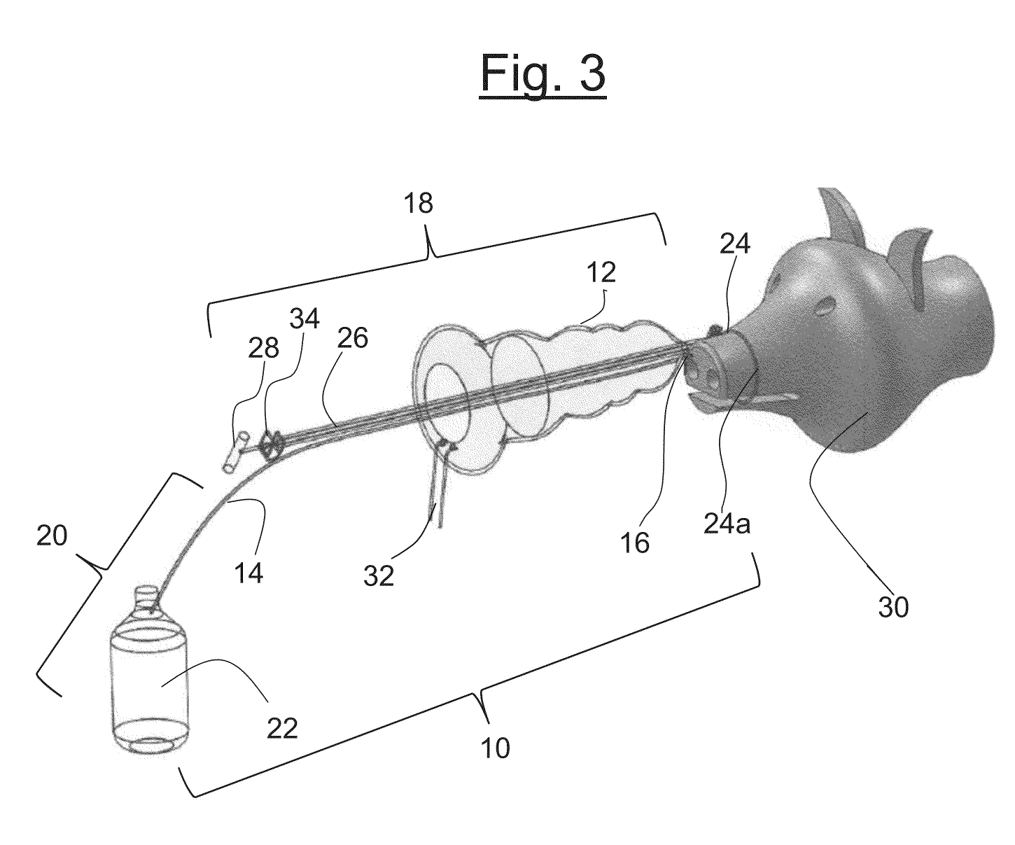

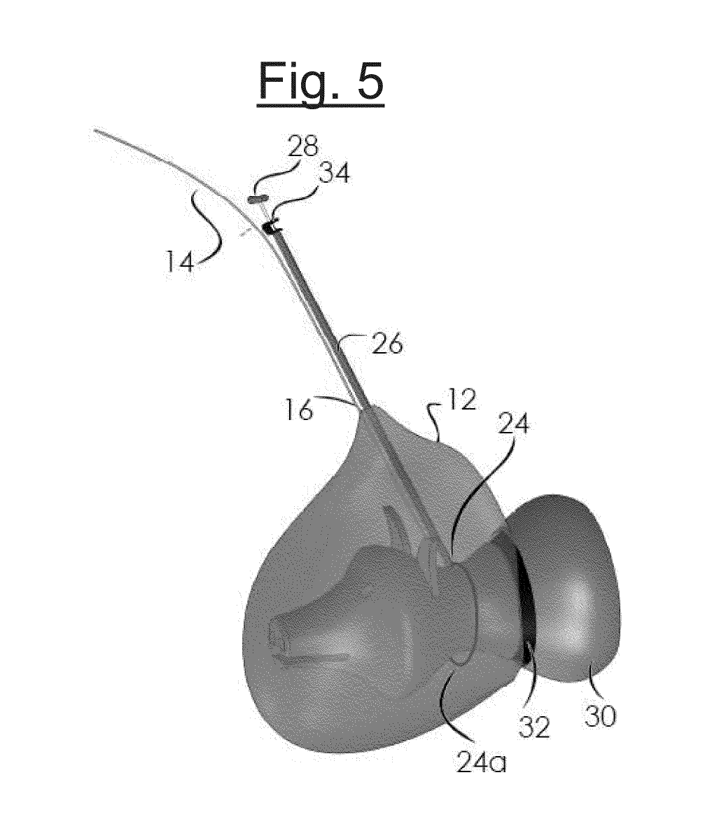

The animal restraint system 18 depicted in the Figures includes a snare cable 24, a pole 26, and a retractor 28. The snare cable 24 may be made of metal, wire, metal fibers, rubber, plastic or other materials. The snare cable 24 may be placed inside the mouth of the animal 30 to be secured, around all or some portion of the snout of the animal 30 as depicted in FIG. 3, or the snare cable 24 may be designed to encircle the neck of the animal 30, to prevent directional movement of the animal 30, as depicted in FIG. 5. Returning to FIG. 3, the snare cable 24 may have a cable loop 24a at the distal end of the snare cable 24, allowing one portion of the cable loop 24a to be placed inside the mouth of the animal 30 and one portion of the cable loop 24a to be placed over the top of the snout of the animal 30, or to allow the cable loop 24a to encircle the outside of the snout of the animal 30. Alternatively, as shown in FIG. 5, the snare cable 24 may have a larger cable loop 24a at the distal end of the snare cable 24 to encircle the head or neck of the animal 30.

The snare cable 24 is operably attached to the pole 26. The pole 26 is at least partially hollow to hold the snare cable 24. As depicted in the Figures, snare cable 24 may be threaded through the length of pole 26.

In use, pole 26 is held by the operator to prevent or minimize movement of the animal 30. As shown in FIG. 2, the snare cable 24 may be tightened by reducing the exposed diameter of the cable loop 24a when the retractor 28 is engaged. The retractor 28 may be locked in the retracted position by a simple lock 34 that prevents the release of the snare cable 24, by applying friction or a grip to the snare cable 24. This allows the operator to place the snare cable 24 into or over the animal's mouth or around the animal's neck or snout and secure the animal 30 by pulling the retractor 28 proximally. The retractor 28 reduces the length of the cable to provide a more secure fit around the animal 30. Animal restraint system 18 depicted in the Figures and described herein is one exemplary embodiment of an animal restraint system. Any system for restraining an animal known in the art, including grippers, pincers, locks, cages or crates, may be used consistent with the present invention.

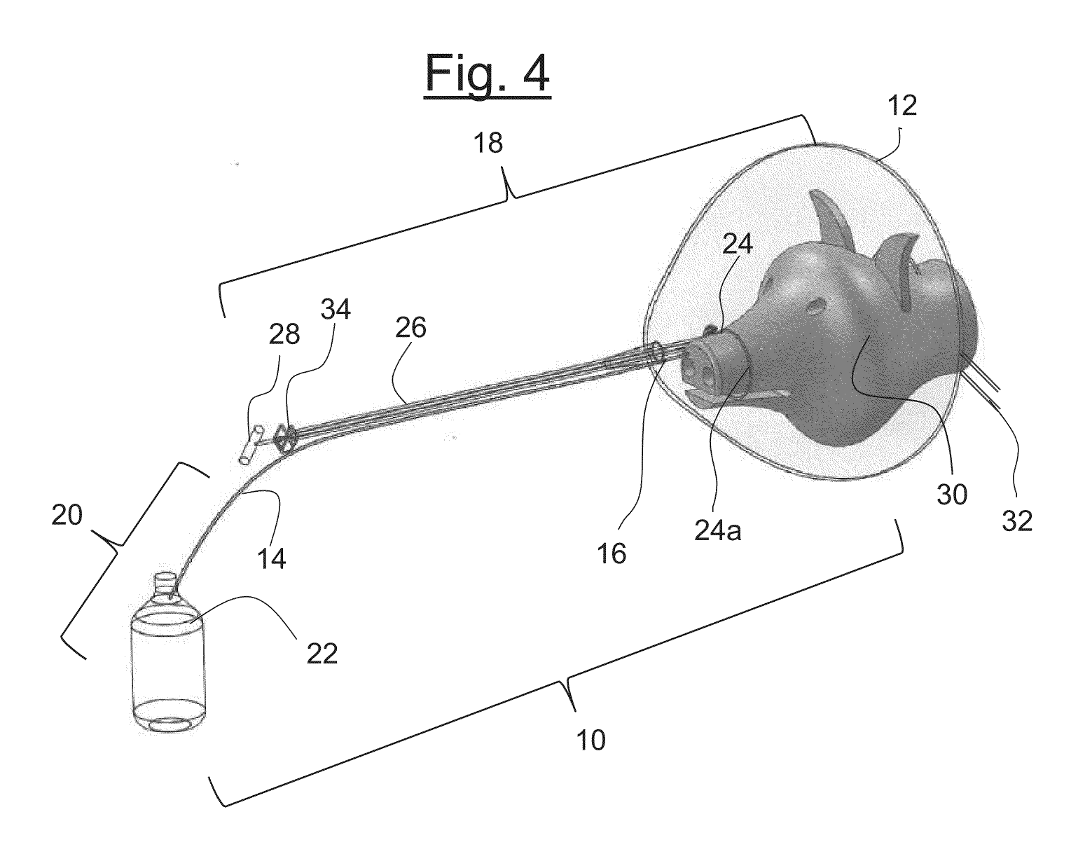

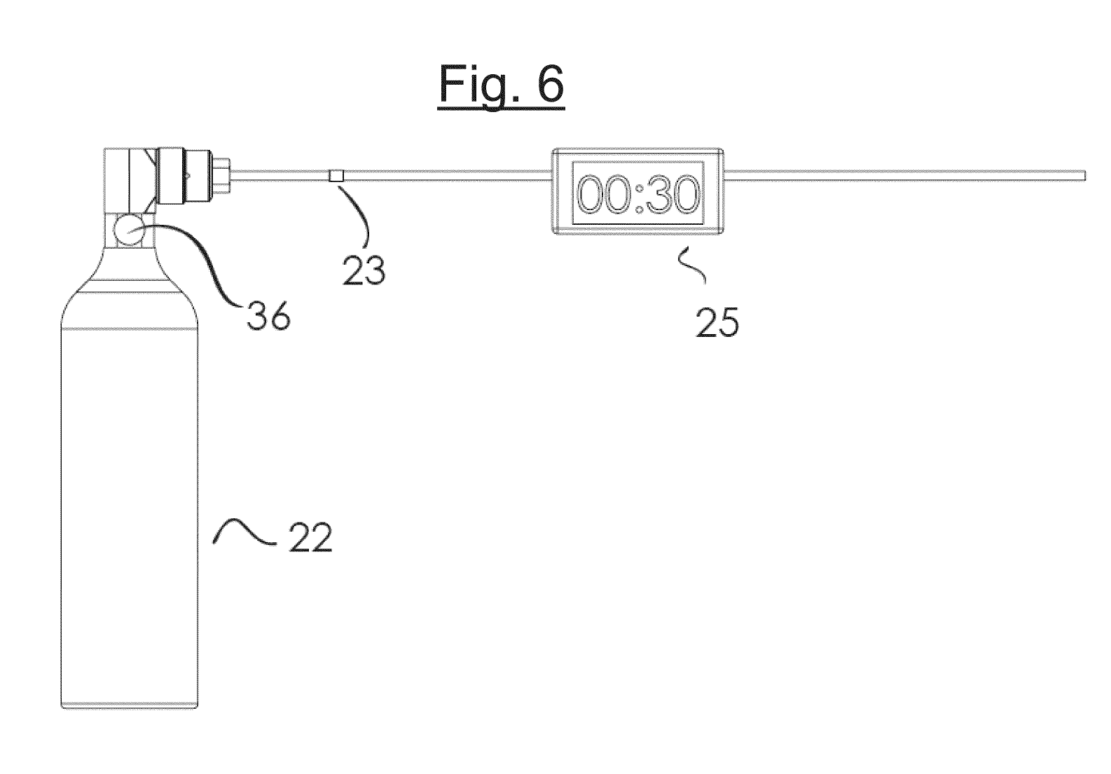

As shown in FIG. 3 and FIG. 4, when the animal 30 is secured by the animal restraint system 18, the hood enclosure 12 is moved from the first open position, which surrounds the pole 26, to the second closed position where it is placed over and surrounds the head of the animal 30. The gas is then supplied from the gas reservoir 22 to the hood enclosure 12 by the gas supply tube 14. In a preferred embodiment, the gas reservoir 22 supplies a metered amount of gas for a specified duration of time to minimize the risk of human error, to ensure that anesthesia or euthanasia are achieved and to prevent wastage of gas. As shown in FIG. 6, the gas supplied from the gas reservoir 22 may be metered by one or more narrow orifices 23 that restrict the volume of gas that flows through the orifice(s) 23 and gas supply tube 14 and into the hood enclosure 12 over a specified period of time. The orifice(s) 23 may be used to increase or decrease the flow rate of the gas supply from gas reservoir 22 by changing the diameter of one or more of the orifices 23, or by bypassing one or more of the orifices 23. As shown in FIG. 6, the gas supply from the gas reservoir 22 may also be metered by time, with a timer 25 that mechanically or electrically stops the gas supply from the gas reservoir 22 after a specified period of time. A valve 36 may be utilized to start and stop the flow of gas from gas reservoir 22 into tube 14. The gas supplied into the animal hood enclosure 12 may be any gas capable of anesthetizing or euthanizing an animal. In the preferred embodiment, when used for euthanasia, the gas supplied into the animal hood enclosure 12 is carbon dioxide or other gas capable of inducing anesthesia and euthanasia.

In a typical embodiment of the present invention, the steps for using the present invention include one or more of the following steps: Engage the animal in the animal restraint system, for example by placing the cable loop 24a that is at the distal end of the animal restraint system 18 inside the mouth of the animal 30; positioning the cable loop over the top portion of the snout of the animal 30, so that the cable loop completely encircles the top portion of the snout of the animal 30; retracting the snare cable 24 by pulling the retractor 28 proximally to tighten the snare cable 24 over the snout of the animal 30 and control the movement of the animal 30; and locking the retractor 28 to prevent an increase or decrease in the exposed length of the snare cable 24. Pull the hood enclosure 12 distally and place it over the head or neck of the animal 30. Open the gas reservoir 22 to allow gas to flow from the gas reservoir 22 into the gas supply tube 14 and into the hood enclosure 12 through the opening 16. Maintain the gas supply in this open position for a specified period of time. Close the gas reservoir 22 to stop the flow of gas from the gas reservoir 22. Pull the hood enclosure 18 proximally to place the hood enclosure 18 in the open position. Release the lock on the retractor 28. Push the retractor 28 distally to loosen and provide slack to the snare cable 24. Remove the animal restraint system 18 from the animal 30.

The present invention has been described in relation to particular embodiments, which are intended in all respects to be illustrative rather than restrictive. Alternative embodiments will become apparent to those of ordinary skill in the art to which the present invention pertains without departing from its scope.

From the foregoing, it will be seen that this invention is one well adapted to attain all the ends and objects set forth above, together with other advantages which are obvious and inherent to the system and method. It will be understood that certain features and sub-combinations are of utility and may be employed without reference to other features and sub-combinations. This is contemplated and within the scope of the claims.

What is claimed is:

1. An inhaled agent administration system for providing a gas to an animal, comprising:
an animal restraint system to minimize animal movement;
a hood enclosure configured to cover the animal's head having a first open position and a second closed position and wherein the hood enclosure is configured to allow unencumbered use of the animal restraint system when in the first open position; and
a gas supply system in fluid communication with the hood enclosure; and
wherein the hood enclosure is configured to restrict release of a gas when the hood enclosure is in the second closed position.

2. The system of claim 1, wherein the hood enclosure is configured to be moved from the first open position to the second closed position without interfering with the animal restraint system.

3. The system of claim 1, wherein the animal restraint system comprises a snare configured to interface with the animal's mouth, snout or both the mouth and snout.

4. The system of claim 1, wherein the animal restraint system comprises a snare configured to interface with the animal's head or neck.

5. The system of claim 1, wherein the gas supply system comprises a gas supply tube opening into the hood enclosure.

6. The system of claim 5, wherein the gas supply system comprises a portable gas tank reservoir and the gas supply tube connects the reservoir to the hood enclosure.

7. The system of claim 1, wherein the gas supply system comprises a timer to control the supply gas for a specified period of time.

8. A method of anesthetizing or euthanizing an animal, the method comprising the steps of:
physically restraining the animal with an animal restraint system;
moving a hood enclosure from a first open position to a second closed position over the animal's head;
supplying a gas into the hood enclosure through a gas supply system in a sufficient amount to anesthetize or euthanize the animal.

9. The method of claim 8 wherein the supplying step comprises supplying the gas for a specified period of time.

10. The method of claim 8, wherein the hood enclosure is attached to the animal restraint system.

11. A method of providing medication to an animal, the method comprising the steps of:
physically restraining the animal with an animal restraint system;
moving a hood enclosure from a first open position to a second closed position over the animal's head;
supplying a gas into the hood enclosure through a gas supply system in a specified amount.
